(19) United States
(12) Patent Application Publication
(10) Pub. No.: US 2023/0159460 A1
(43) Pub. Date: May 25, 2023

(54) STABILIZATION OF THERMOSET COMPOSITES AND COATING WITH A UV STABILIZING TECHNOLOGY AND TECHNIQUE FOR PRODUCING SAME

(71) Applicant: Chromaflo Technologies Corporation, Ashtabula, OH (US)

(72) Inventors: Santosh K. Yadav, Geneva, OH (US); Paul A. Rettinger, Ashtabula, OH (US)

(21) Appl. No.: 18/057,814

(22) Filed: Nov. 22, 2022

Related U.S. Application Data
(60) Provisional application No. 63/264,468, filed on Nov. 23, 2021.

(51) Int. Cl. C07D 211/46; C08K 5/3435

(52) U.S. Cl. CPC C07D 211/46; C08K 5/3435

(57) ABSTRACT

A reaction scheme to synthesize high functional UVS molecules such as 1,2,2,6,6-pentamethylpiperidin-4-yl methacrylate and use 1,2,2,6,6-pentamethylpiperidin-4-yl methacrylate for improved UV stability of a product using UVS additives.

### RELATED APPLICATIONS

This claims the benefit of U.S. Provisional Application No. 63/264,468, filed Nov. 23, 2021, which application is incorporated herein by reference in its entirety.

### BACKGROUND

Without UV stabilization package, unsaturated polymer composites or coating undergo significant failure within several hundred hours of accelerated UV exposure. There are several degradation mechanisms have been proposed for these failure modes. Most common is photo-oxidation of polymers contributes to oxidative yellowing. Long time exposure of UV light creates micro cracks through chain scission. Surface cracking may also relate to gradient stress in degraded panels. High UV radiation may initiate phototropic post-crosslinking, resulting in interfacial tension, and further builds up stress in materials from the surface to the inner layers.

To protect the effect of UV degradation, INA (ultraviolet absorbers) additives need to be added in the formulations. UV stability to prevent UV-induced polymer degradations when exposed to sunlight and therefore have a longer service life than hose without any coating.

SUMMARY

We now describe a reaction scheme for production of a piperidinyl acrylate to synthesize high functional UV stabilizing (“UVS”) molecules such as 1,2,2,6,6-pentamethylpiperidin-4-yl methacrylate for improved UV stability of a product using UVS additives for enhanced UV or outdoor weatherability.

The reaction schemes includes steps of forming a reaction mixture of 1,2,2,6,6-Pentamethyl-4-piperidinol, 4-dimethylaminopyridine and methacrylic anhydride at a temperature between about −20° C. and 25° C., warming the reaction mixture to room temperature, and reacting the reaction mixture for 18 hours. The reaction mixture is quenched with aqueous saturated NaHCO₃ and a 1,2,2,6,6-pentamethylpiperidin-4-yl methacrylate product is purified. The 1,2,2,6,6-pentamethylpiperidin-4-yl methacrylate product is characterized by having an FTIR spectra of FIG. 2D or having an GC-MS spectra of FIG. 3.

In one aspect, the invention includes a UV stabilizing additive package that has 1,2,2,6,6-pentamethylpiperidin-4-yl methacrylate as part of the additive package.

In one aspect, the thermoset composite may include 1,2,2,6,6-pentamethylpiperidin-4-yl methacrylate in an amount of about 2.2 phr.

Other methods, features and/or advantages is, or will become, apparent upon examination of the following figures and detailed description. It is intended that all such additional methods, features, and advantages be included within this description and be protected by the accompanying claims.

### BRIEF DESCRIPTION OF THE DRAWINGS

In the accompanying figures, chemical formulas, chemical structures, and experimental data are given that, together with the detailed description provided below, describe example embodiments of the claimed invention.

FIG. 1A-1B are reaction schemes for production of a piperidinyl acrylate (1A) and 1,2,2,6,6-pentamethylpiperidin-4-yl methacrylate (1B) according to aspects of the invention.

FIG. 2A-2D are FTIR spectra demonstrating formation of 1,2,2,6,6-pentamethylpiperidin-4-yl methacrylate from the starting material of methacrylic anhydride. FIG. 2A a FTIR spectra of methacrylic anhydride, FIGS. 2B a FTIR spectra of 1,2,2,6,6-pentamethyl-4-piperidinol; FIG. 2C an FTIR spectra of initial mix of methacrylic anhydride, 1,2,2,6,6-pentamethylpiperidinol, and DMAP at start of reaction; and FIG. 2D a FTIR spectra of the 1,2,2,6,6-pentamethylpiperidin-4-yl methacrylate, completed reaction.

FIG. 3 is GC-MS analysis of 1,2,2,6,6-pentamethylpiperidin-4-yl methacrylate.

FIG. 4 is a GC-MS overlay demonstrating reproducibility of 1,2,2,6,6-pentamethylpiperidin-4-yl methacrylate produced according to one aspect of the invention.

FIG. 5A-5C are weathering data of three thermoset composites prepared according to one aspect of the invention.

### DETAILED DESCRIPTION

Certain aspects are described below. While the embodiments are described in considerable detail, it is not the intention to restrict or in any way limit the scope of the appended claims to such detail, or to any particular embodiment.

### Definitions

As used herein, the term “organic group” is used to mean a hydrocarbon group that is classified as an aliphatic group, cyclic group, or combination of aliphatic and cyclic groups (e.g., alkaryl and aralkyl groups). In the context of the present invention, suitable organic groups for the compounds of this invention are those that do not interfere with the anti-aging activity of the compounds. In the context of the present invention, the term “aliphatic group” means a saturated or unsaturated linear or branched hydrocarbon group. This term is used to encompass alkyl, alkenyl, and alkynyl groups, for example.

As used herein the term hydrocarbyl is inclusive of a number of carbon atoms in any configuration. For example a C₆ hydrocarbyl group comprises alkyl, aryl and cycloalkyl configurations. The carbon atoms of the hydrocarbyl group may be saturated or unsaturated.

As used herein, the terms “alkyl”, “alkenyl”, and the prefix “alk-” are inclusive of straight chain groups and branched chain groups. Unless otherwise specified, these groups contain from 1 to 20 carbon atoms, with alkenyl groups containing from 2 to 20 carbon atoms. In some embodiments, these groups have a total of at most 10 carbon atoms, at most 8 carbon atoms, at most 6 carbon atoms, or at most 4 carbon atoms. Alkyl groups including 4 or fewer carbon atoms can also be referred to as lower alkyl groups. Alkyl groups can also be referred to by the number of carbon atoms that they include (i.e., C₁-C₄ alkyl groups are alky groups including 1-4 carbon atoms).

Cycloalkyl, as used herein, refers to an alkyl group (i.e., an alkyl, alkenyl, or alkynyl group) that forms a ring structure. Cyclic groups can be monocyclic or polycyclic and preferably have from 3 to 10 ring carbon atoms. A cycloalkyl group can be attached to the main structure via an alkyl group including 4 or less carbon atoms. Exemplary cyclic groups include cyclopropyl, cyclopropylmethyl, cyclopentyl, cyclohexyl, adamantyl, and substituted and unsubstituted bornyl, norbornyl, and norbornenyl.

Unless otherwise specified, “alkylene” and “alkenylene” are the divalent forms of the “alkyl” and “alkenyl” groups defined above. The terms, “alkylenyl” and “alkenylenyl” are used when “alkylene” and “alkenylene”, respectively, are substituted. For example, an arylalkylenyl group comprises an alkylene moiety to which an aryl group is attached.

The term “aryl” as used herein includes carbocyclic aromatic rings or ring systems. Examples of aryl groups include phenyl, naphthyl, biphenyl, fluorenyl and indenyl. Aryl groups may be substituted or unsubstituted.

When a group is present more than once in any formula or scheme described herein, each group (or substituent) is independently selected, whether explicitly stated or not. For example, for the formula —C(O)—NR₂ each R group is independently selected.

As a means of simplifying the discussion and the recitation of certain terminology used throughout this application, the terms “group” and “moiety” are used to differentiate between chemical species that allow for substitution or that may be substituted and those that do not so allow for substitution or may not be so substituted. Thus, when the term “group” is used to describe a chemical substituent, the described chemical material includes the unsubstituted group and that group with nonperoxidic O, N, S, Si, or F atoms, for example, in the chain as well as carbonyl groups or other conventional substituents. Where the term “moiety” is used to describe a chemical compound or substituent, only an unsubstituted chemical material is intended to be included. For example, the phrase “alkyl group” is intended to include not only pure open chain saturated hydrocarbon alkyl substituents, such as methyl, ethyl, propyl, tert-butyl, and the like, but also alkyl substituents bearing further substituents known in the art, such as hydroxy, alkoxy, alkylsulfonyl, halogen atoms, cyano, nitro, amino, carboxyl, etc. Thus, “alkyl group” includes ether groups, haloalkyls, nitroalkyls, carboxyalkyls, hydroxyalkyls, cyanoalkyls, etc. On the other hand, the phrase “alkyl moiety” is limited to the inclusion of only pure open chain saturated hydrocarbon alkyl substituents, such as methyl, ethyl, propyl, tert-butyl, and the like.

To the extent that the term “includes” or “including” is used in the specification or the claims, it is intended to be inclusive in a manner similar to the term “comprising” as that term is interpreted when employed as a transitional word in a claim. Furthermore, to the extent that the term “or” is employed (e.g., A or B) it is intended to mean “A or B or both.” When “only A or B but not both” is intended, then the term “only A or B but not both” will be employed. Thus, use of the term “or” herein is the inclusive, and not the exclusive use. As used in the specification and the claims, the singular forms “a,” “an,” and “the” include the plural. Finally, where the term “about” is used in conjunction with a number, it is intended to include ±1.0% of the number. For example, “about 10” may mean from 9 to 11. Reactant and component refer to the same concept and refer to part of the reactant mixture as a whole. The term film could also refer to a coating or sheet or layer that is applied to a surface. The surface may be any desired material or shape.

In one aspect, the invention fully describes a process for preparation of any piperidinyl acrylate, and in particular 1,2,2,6,6-pentamethylpiperidin-4-yl methacrylate. Briefly, a reaction mixture of 1,2,2,6,6-Pentamethyl-4-piperidinol, 4-dimethylaminopyridine and methacrylic anhydride at a temperature between about −20° C. and 10° C. is formed. After warming the reaction mixture to room temperature (18° C.-25° C.), and reacting the reaction mixture for 18 (between 15 and 24) , the reaction mixture is quenched with aqueous saturated NaHCO₃ and a 1,2,2,6,6-pentamethylpiperidin-4-yl methacrylate product is purified.

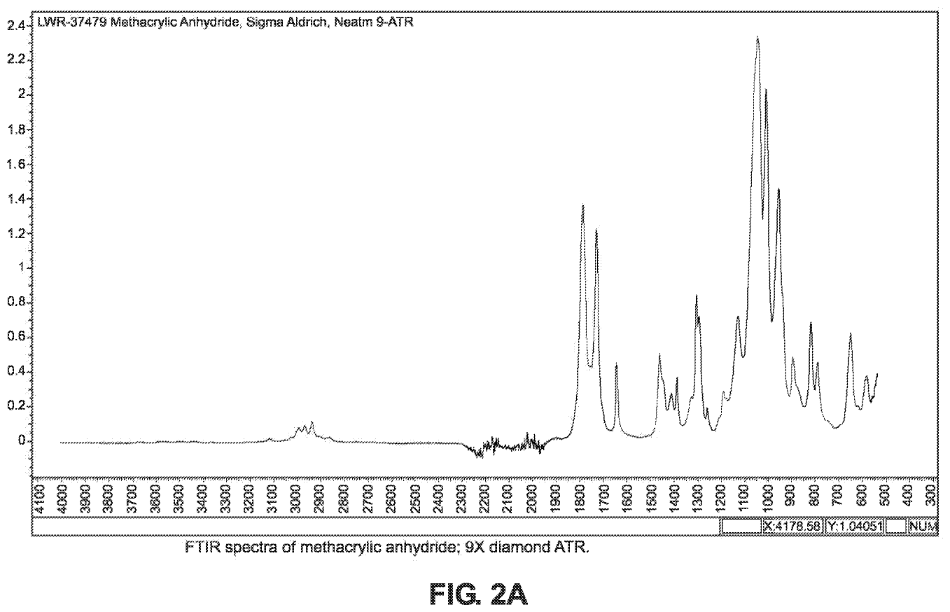

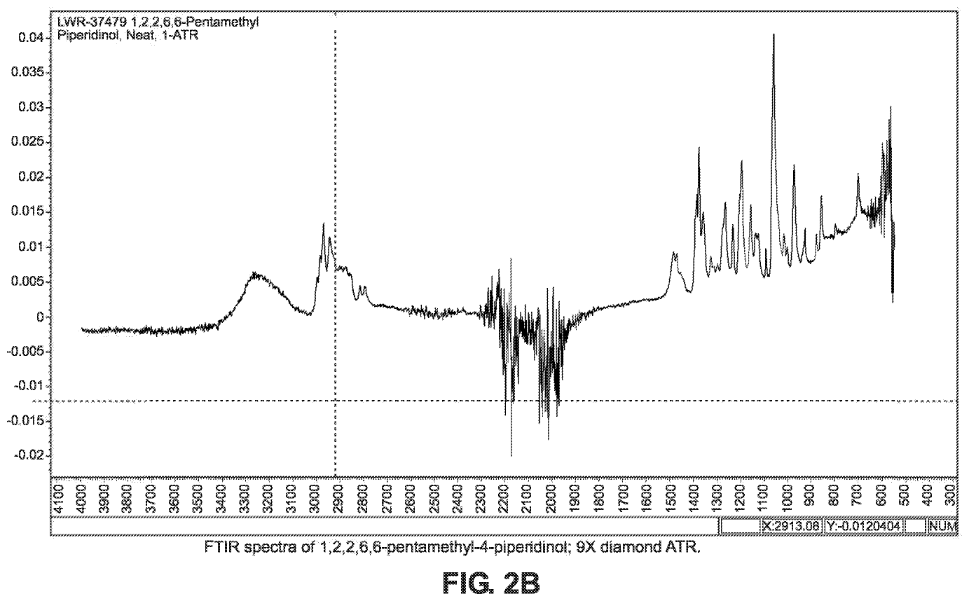

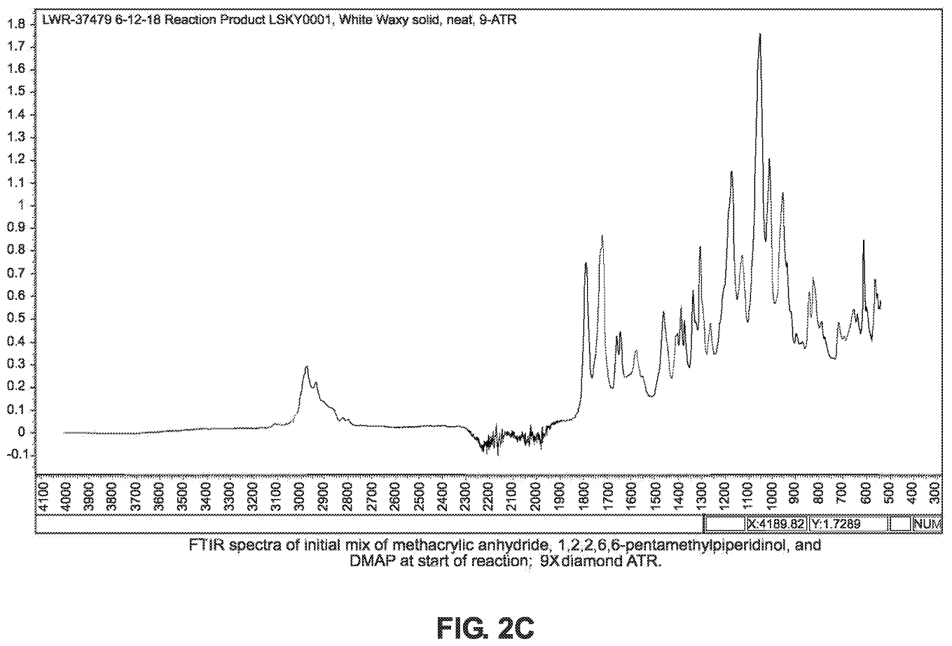

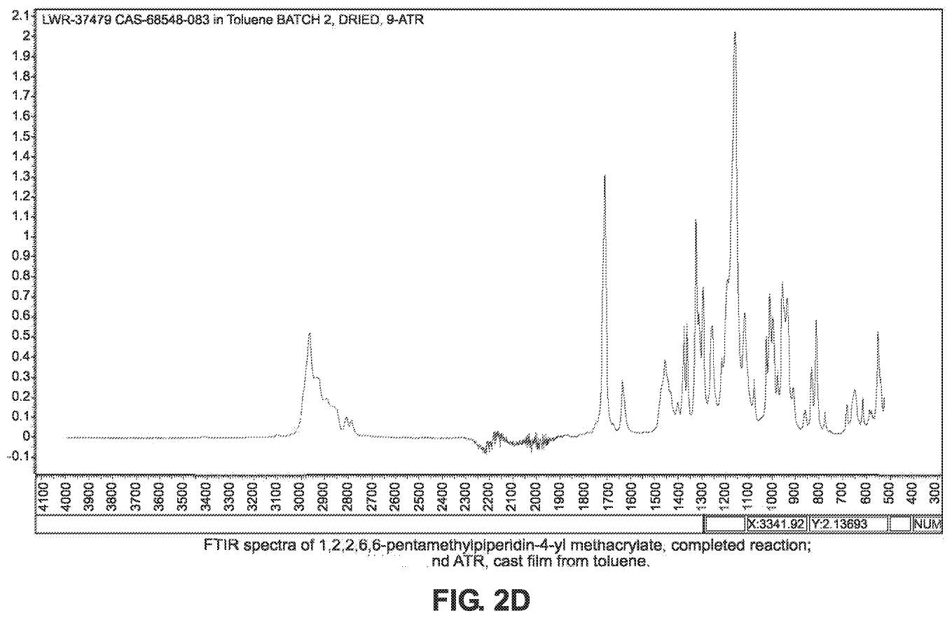

In one aspect, the 1,2,2,6,6-pentamethylpiperidin-4-yl methacrylate product is characterized by having an FTIR spectra of FIG. 2D.

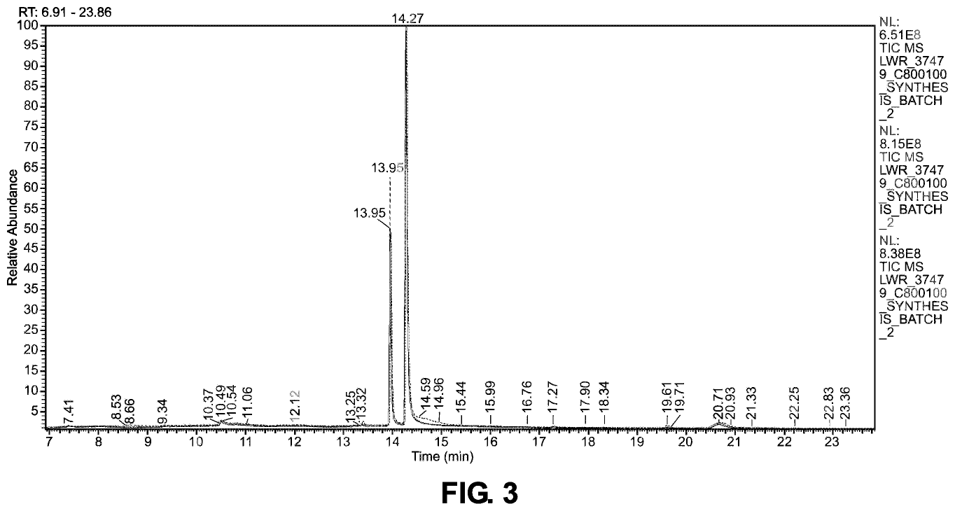

In one aspect, the 1,2,2,6,6-pentamethylpiperidin-4-yl methacrylate product is characterized by having an GC-MS spectra of FIG. 3.

In one aspect, the 1,2,2,6,6-pentamethylpiperidin-4-yl methacrylate reaction scheme includes a step of forming a reaction mixture with 1,2,2,6,6-Pentamethyl-4-piperidinol (50.0 g, 292 mmol), 4-dimethylaminopyridine (4-DMAP, 3.57 g, 29.2 mmol) and methacrylic anhydride (45 g, 292 mmol).

In one aspect, the 1,2,2,6,6-pentamethylpiperidin-4-yl methacrylate reaction scheme includes forming the reaction mixture at about 0° C. or at 0° C.

In one aspect, the step of purifying a 1,2,2,6,6-pentamethylpiperidin-4-yl methacrylate product from the reaction mixture may include: stirring the quenched reaction product for about 30 minutes; partitioning the layers and extracting a solution comprising the product; sequentially washing the solution with aqueous saturated NaHCO₃ (2×300 mL), water (300 mL), brine (300 mL); drying the washed solution over MgSO₄; and filtering off salts to obtain a purified 1,2,2,6,6-pentamethylpiperidin-4-yl methacrylate product.

In one aspect, the purified 1,2,2,6,6-pentamethylpiperidin-4-yl methacrylate product is 97% active.

In one aspect, the invention includes a UV stabilizing additive package that has 1,2,2,6,6-pentamethylpiperidin-4-yl methacrylate as part of the additive package.

In one aspect, the 1,2,2,6,6-pentamethylpiperidin-4-yl methacrylate of the additive package is characterized by having an FTIR spectra of FIG. 2D.

In one aspect, the 1,2,2,6,6-pentamethylpiperidin-4-yl methacrylate of the additive package is characterized by having a GC-MS spectra of FIG. 3.

In one aspect, the invention describes a thermoset composite comprising 1,2,2,6,6-pentamethylpiperidin-4-yl methacrylate.

In one aspect, the thermoset composite may include 1,2,2,6,6-pentamethylpiperidin-4-yl methacrylate in an amount between about 0.1 phr and 5 phr, or in an amount of about 2.2 phr, or in an amount of 2.2 phr or 0.8 wt % of molded part.

In one aspect, the 1,2,2,6,6-pentamethylpiperidin-4-yl methacrylate of a thermoset composite is prepared by: forming a reaction mixture of 1,2,2,6,6-Pentamethyl-4-piperidinol, 4-dimethylaminopyridine and methacrylic anhydride at a temperature between about −20° C. and 10° C.; warming the reaction mixture to room temperature (18° C.-25° C.); reacting the reaction mixture for 18 (between 15 and 24) hours; quenching the reaction with aqueous saturated NaHCO₃; and purifying a 1,2,2,6,6-pentamethylpiperidin-4-yl methacrylate product from the quenched reaction mixture.

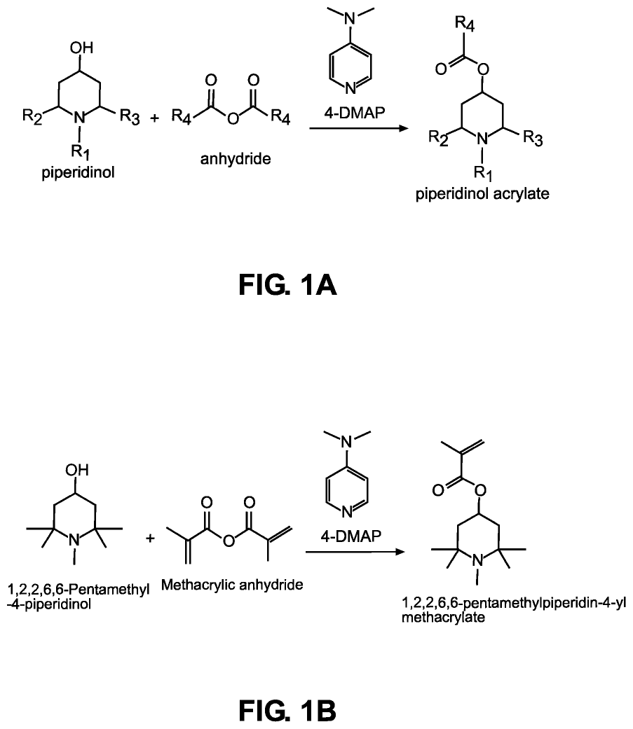

A reaction scheme for production of a piperidinyl acrylate is shown in FIG. 1A. Briefly a piperidinol of the general formula:

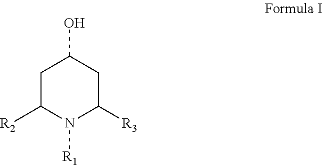

where R₁, R₂, and R₃ are each independently hydrogen; a C₁ to C₆ hydrocarbyl group; a C₁ to C₆ aromatic, alkyl, aryl or alkoxy group, a C₁ to C₆ hydrocarbyl group, or a substituted or unsubstituted straight chain C₅ to C₂₄ alkyl group, or an unsubstituted straight chain C₅ to C₁₂ alkyl group is combined with an anyhydride of the general formula:

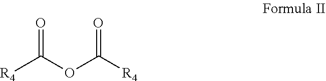

where each R₄ is C₁ to C₆ hydrocarbyl group; a C₁ to C₆ alkyl, aryl or alkoxy group or a C₁ to C₆ hydrocarbyl group, or an unsubstituted straight chain C₅ to C₁₂ alkyl group, a saturated or unsaturated hydrocarbon that is C₆-C₂₄ in length.

The reaction scheme producing a product shown in Formula III:

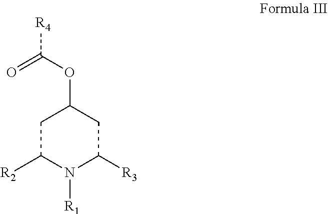

according to the reaction scheme of FIG. 1A.

The reaction scheme of FIG. 1A, when applied to the reactants 1,2,2,6,6-pentamethyl-4-piperidinol and methacrylic anhydride produces a product of formula IV:

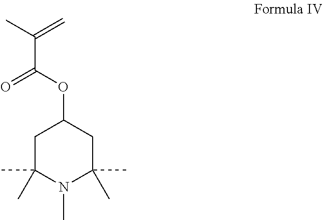

Referring specifically to the reaction scheme, a piperidinol and anhydride reactants are combined at a temperature that is preferably near 0° C. While the temperature may vary between −20° C. and 25° C. , or between −10° C. and 10° C., or between −5° C. and 5° C. or more preferably between −1° C. and 1° C., it is appreciated that any single specific temperature within any of these ranges is encompassed by the invention.

Piperidinol reactant: Generally any reactant with the general structure of Formula I is usable in the processes of the invention. Further, it is understood that while the reaction schemes are exemplified by the use of a 4-piperidinol. Piperidinols characterized as a 3-piperidinol, a 2-piperidinol, or a 1-piperidonol are additionally also encompassed as an aspect of the instant invention.

Anhydride reactant: Generally any anhydride of the general structure of Formula II is usable in the processes of the invention. In some cases, the anhydride is an acrylic anhydride, a methacrylic anhydride, or an isobutacrylic anhydride, a maelic anhydride, a butyric anhydride, a hexanoic anhydride, a cyclohexanecarboxylic anhydride, a propionic anhydride, an ethanoic anhydride, an acetic anhydride, a butanoic anhydride, a saturated or unsaturated hydrocarbon that is C₆-C₂₄ in length, or any organic acid anhydride.

In addition to the reactants, a catalyst is added to form a reactant mixture. In some cases the catalyst is 4-DMAP. The piperidinol, anhydride, catalyst reactant mixture is permitted to rise from the temperature near 0° C. to room temperature. Room temperature may be defined as between about 20° C. and 22° C. It is appreciated though that room temperature may in fact comprise a temperature range between about 18° C. and 25° C. Attainment of room temperature may be the result of maintaining the reaction mixture in a stable room temperature environment for a sufficient period of time to attain room temperature through equilibrium or may be the result of application of heat from an external source to the reaction mixture.

Preferably the piperidinol, anhydride, catalyst reactant mixture is maintained at room temperature for a reaction time of about 18 hours. It is appreciated that the reaction time may vary from about 15 hours to about 24 hours and includes any single numerical value found within this range. The reaction time may be measured from the point the reactant mixture is combined or alternatively timing may commence once the reactant mixture attains room temperature.

After the reaction time is completed, the reaction is quenched. An exemplary quenching agent is saturated NaHCO₃. However, it is appreciated that additional or difference quenching agents are well within the scope of the invention. If saturated NaHCO₃ is added to the reactant mixture, for example, the quenched reaction mixture is stirred, partitioned, and the solution comprising the reactant product is washed with saturated NaHCO₃, water, brine. Followed by drying over MGSO₄ and filtering off salts.

The reaction scheme described has several advantages. Not the least of which is that high reaction temperatures are avoided which tend to volatilize and cause polymerization of the anhydride reactant. Additionally expensive and time consuming processes and the use of salts are unnecessary with this reaction scheme.

The reactant product, a piperidinyl acrylate, and specifically 1,2,2,6,6-pentamethylpiperidin-4-yl methacrylate is an effective UV stabilizer additive. The UV stabilizer may include 1,2,2,6,6-pentamethylpiperidin-4-yl methacrylate or a blend of 1,2,2,6,6-pentamethylpiperidin-4-yl methacrylate and other UV stabilizers.

### EXAMPLES

### Example 1: Synthesis of 1,2,2,6,6-pentamethylpiperidin-4-yl methacrylate

Materials:
a) 1,2,2,6,6-Pentamethyl-4-piperidinol;
b) 4-dimethylaminopyridine (4-DMAP);
c) Methacrylic anhydride; and
d) Sodium bicarbonate

Equipment:
a) Round bottle flask;
b) Magnetic stirrer hot plate;
c) Separatory funnel; and
d) Glass reflux coil condenser

Procedure:

1,2,2,6,6-pentamethyl-4-piperidinol (50.0 g, 292 mmol) and 4-dimethylaminopyridine (4-DMAP, 3.57 g, 29.2 mmol) and methacrylic anhydride (45 g, 292 mmol) are added in round bottom flask at lower temperature (0° C., or between about −20° C. and about 10° C.). The reaction mixture is slowly warmed to room temperature (between about 18° C. and 25° C.) and allowed to react for 18 (between about 15-24) hours before quenching with aqueous saturated NaHCO₃ (300 mL) and then stirred vigorously for 30 minutes before partitioning the layers. The solution was sequentially washed with aqueous saturated NaHCO₃ (2×300 mL), water (300 mL), brine (300 mL), dried over MgSO₄ and filtered off the salts.

Characterization of product by FTIR and GC-MS:

In FIG. 2A, FTIR spectra of methacrylic anhydride is known to possess a significant carbonyl absorption at 1780 cm⁻¹ in the infrared spectrum. See FIG. 2A for a characterization of methacrylic anhydride as supplied by Sigma-Aldrich. In the reaction described in FIG. 1, the carbonyl is consumed as methacrylic anhydride reacts with the hydroxyl of 1,2,2,6,6-pentamethyl-4-piperidinol to form an ester. FTIR spectra of 1,2,2,6,6-pentamethyl-4-piperidinol is shown in FIG. 2B. As the reaction progresses, there will be a reduction in the intensity of the anhydride carbonyl at 1780 cm⁻¹(FIG. 2B). As shown in FIG. 2C, the carbonyl absorption at 1780 cm⁻¹ is less intense than a similar carbonyl at 1720 cm⁻¹. FIG. 2D demonstrates the complete removal of the 1780 cm⁻¹, thereby demonstrating a completely reacted product, 1,2,2,6,6-pentamethylpiperidin-4-yl methacrylate.

In FIG. 3, a GC-MS analysis of the completed reacted product, of 1,2,2,6,6-pentamethylpiperidin-4-yl methacrylate is demonstrated.

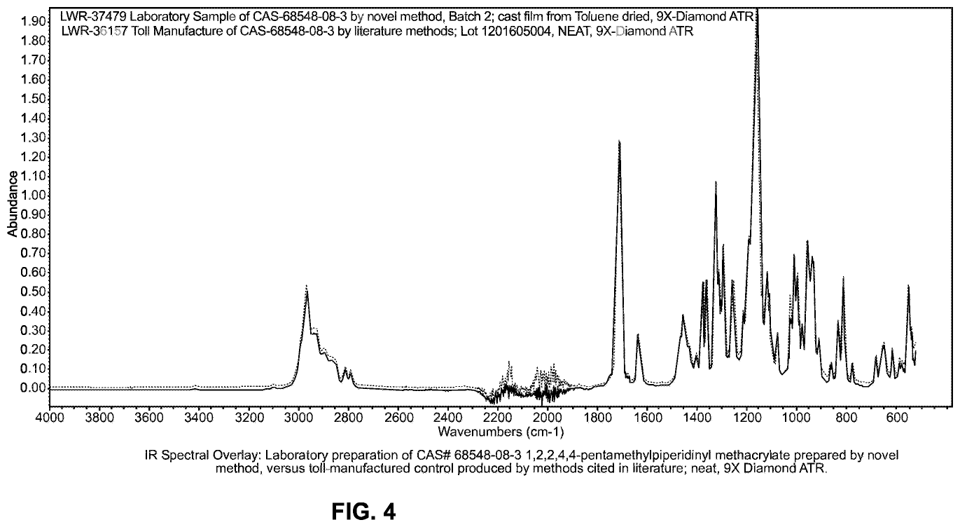

In FIG. 4 the reproducibility of the reaction scheme and purify of the reacted product are demonstrated by a GC-MS overlay indicating identical outputs for different products.

### Example 2: Synthesis of Oleic-Piperidinol Acrylate

Oleic-piperdinyl acrylate may be produced according to the reaction scheme of FIG. 1A. Briefly a piperdinol, 4-dimethylaminopyridine and an oleic anhydride are added in round bottom flask at a temperature of about 0° C. (−20-10). The reaction mixture is slowly warmed to room temperature (18-25° C.) and allowed to react for 18 (about 15 to 24) hours before quenching. Quenching may occur for example with the addition of an aqueous saturated NaHCO₃ (300 mL), stirring vigorously for 30 minutes before partitioning the layers. The solution then sequentially washed with aqueous saturated NaHCO₃ (2×300 mL), water (300 mL), brine (300 mL), dried over MgSO₄ and filtered off the salts.

### Example 3: Weathering Date of a Fiber Reinforced Composite Comprising 1,2,2,6,6-pentamethylpiperidin-4-yl methacrylate

The effectiveness of the 1,2,2,6,6-pentamethylpiperidin-4-yl methacrylate as a UV stabilizer additive was demonstrated by weathering testing.

Black panels were molded using a base bulk molding compound (BMC) formulation comprised of:
i. Polynt Polylite 31610—this is an unsaturated isophthalic-modified polyester resin, 66.5% (w/w) non-volatile resin in 33.5% styrene solution.
ii. Ineos Aropol 63004—this a solution of 66.5% (w/w) polystyrene in 33.5% styrene.
iii. CM-20540 is a dispersion of carbon black in unsaturated polyester resin made by Chromaflo Techologies Corp.
iv. Huber SB 432 is a grade of aluminum trihydrate filler made by Huber Corporation.
v. Synermix 77-90517 is a proprietary mold release formulation made by Chromaflo Technologies Corp.
vi. AM-9033 is a dispersion of 40% magnesium oxide in unsaturated polyester resin made by Chromaflo Technologies Corp.
vii. Experimental additives at 2.2 phr (parts per hundred resin, 0.8% of molded part) described in the Table below.

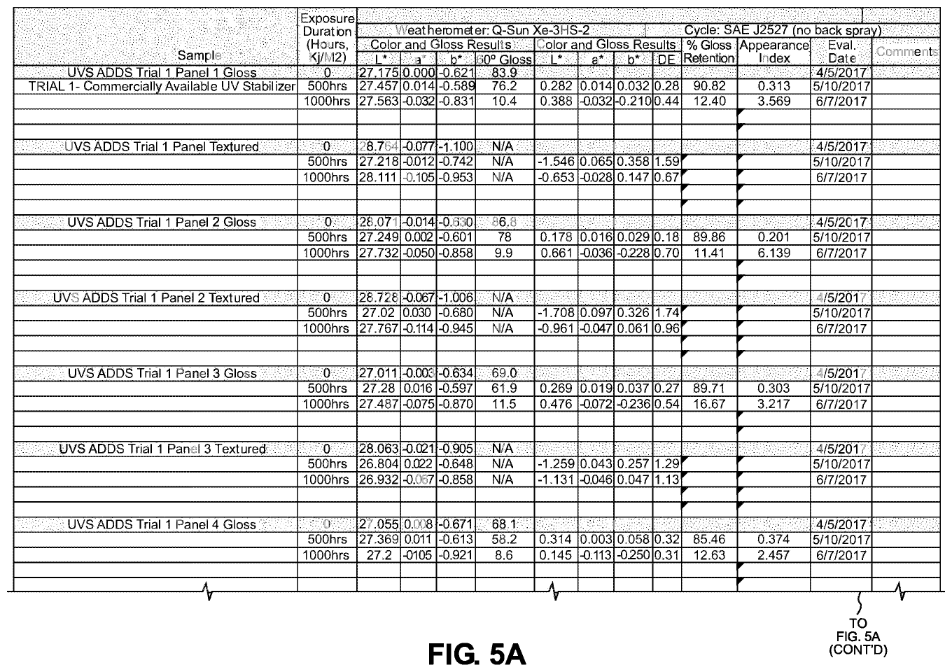

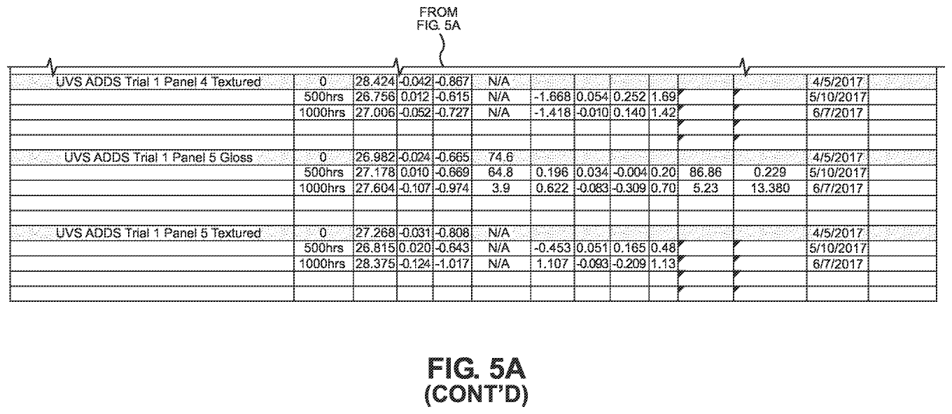

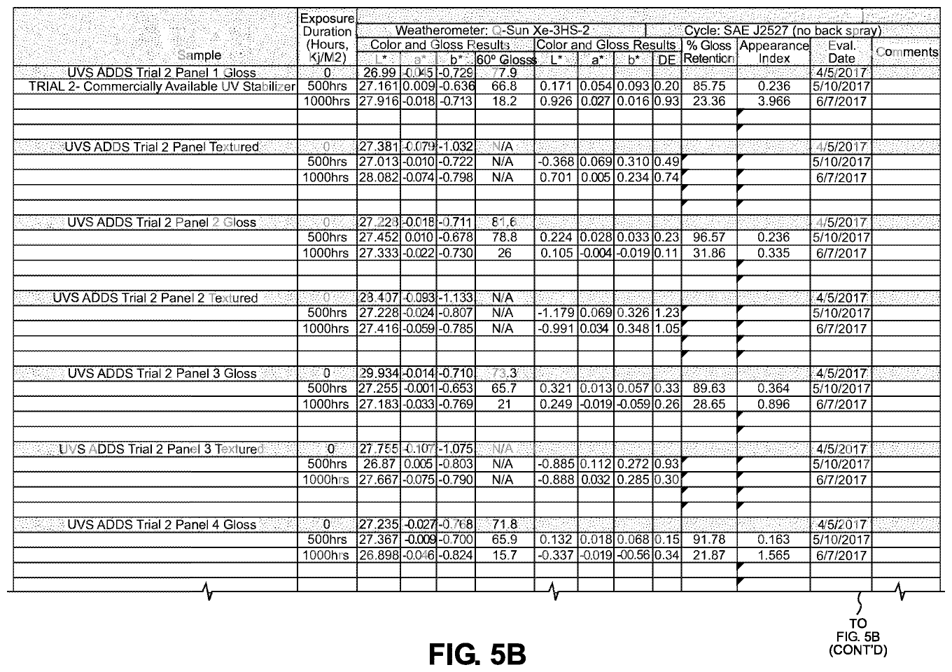

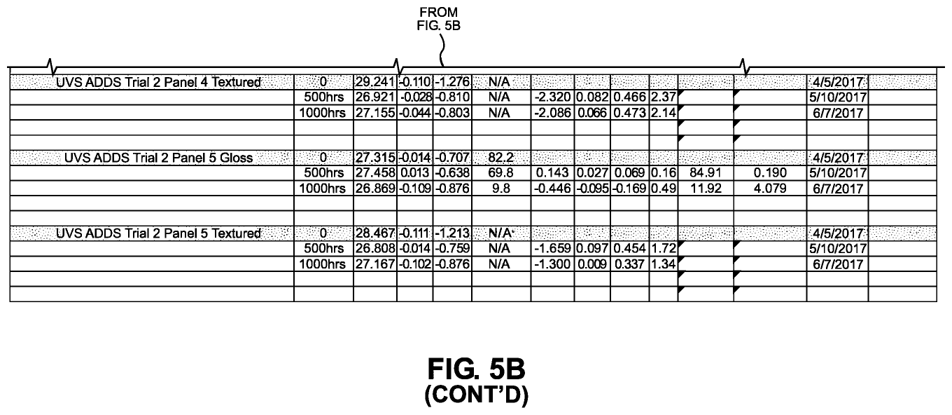

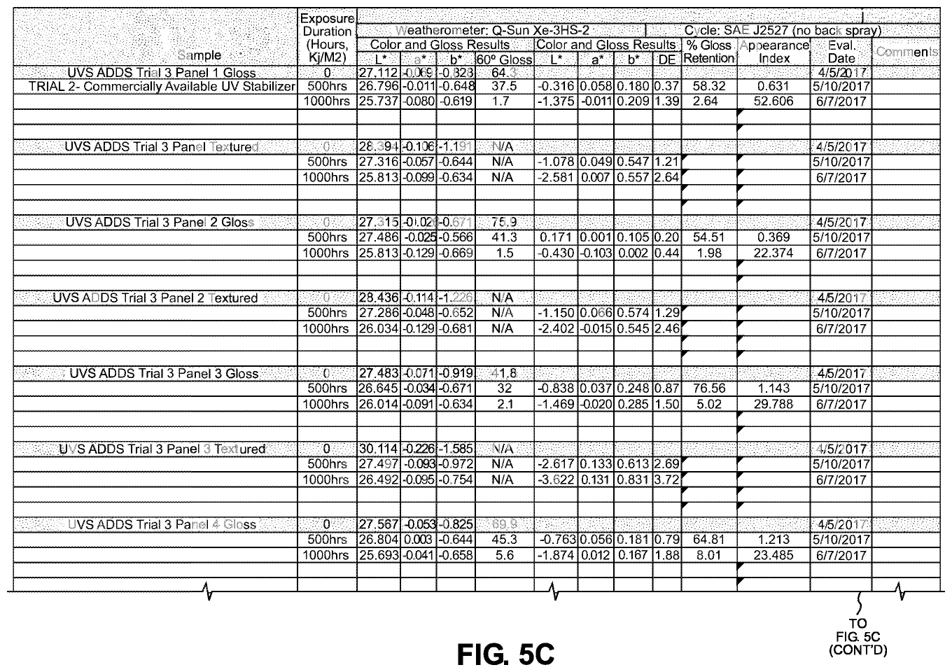

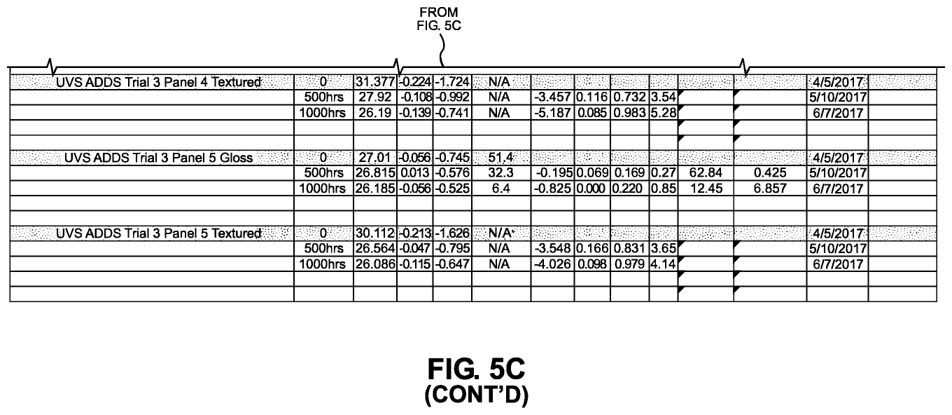

The following experimental additives were added:

| Trial | |
|---|---|
| Number | Additive |
| 1 (FIG. 5A) | Commercially Available UV stabilizer; BASF Tinuvin 292 |
| | or alternate source(s) for same |
| 2 (FIG. 5B) | 1,2,2,6,6-pentamethylpiperidin-4-yl methacrylate |
| 3 (FIG. 5C) | None, Control |

A total of thirty (30) panels, five (5) of each experimental trial were weathered for 1000 hours by ASTM G155 (boro/boro, WR65M), under LWR-36476, WE-70785. With the following % gloss retention at 1000 hours

| | | % gloss |
|---|---|---|
| Trial | | retention at |
| Number | Additive | 1000 hours |
| 1 | Commercially Available UV stabilizer | 11.7% |
| 2 | 1,2,2,6,6-pentamethylpiperidin-4-yl methacrylate | 23.5% |
| 3 | None, Control | 6.02% |

As demonstrated 1,2,2,6,6-pentamethylpiperidin-4-yl methacrylate is particularly effective as a UV stabilizer. Further blends of 1,2,2,6,6-pentamethylpiperidin-4-yl methacrylate with other additives are also effective UV stabilizers.

As stated above, while the present application has been illustrated by the description of embodiments, and while the embodiments have been described in considerable detail, it is not the intention to restrict or in any way limit the scope of the appended claims to such detail. Additional advantages and modifications will readily appear to those skilled in the art, having the benefit of this application. Therefore, the application, in its broader aspects, is not limited to the specific details and illustrative examples shown. Departures may be made from such details and examples without departing from the spirit or scope of the general inventive concept.

1. A process for preparation of a piperidinyl acrylate comprising:
forming a reaction mixture of a piperidinol and an anhydride at a temperature between about −20° C. and 10° C. and a catalyst;
warming the reaction mixture to between about 20° C. and 22° C.;
reacting the reaction mixture for between about 15 hours and 24 hours;
quenching the reaction with aqueous saturated NaHCO₃;
purifying a piperidinyl acrylate product from the quenched reaction mixture.

2. The process of claim 1, wherein the warming of the reaction mixture comprising maintaining the reaction mixture at ambient room temperature to for a period of time sufficient to attain a temperature of between about 20° C. and 22° C., or alternatively application of a heat source to the reaction mixture.

3. The process of claim 1, wherein the piperidinol is a 4-piperidinol, 3-piperidinol, 2-piperidinol, or 1-piperidonol.

4. The process of claim 1, wherein the anhydride is acrylic anhydride, a methacrylic anhydride, or an isobutacrylic anhydride, maelic anhydride, butyric anhydride, hexanoic anhydride, cyclohexanecarboxylic anhydride, propionic anhydride, ethanoic anhydride, acetic anhydride, butanoic anhydride, a saturated or unsaturated hydrocarbon that is C₆-C₂₄ in length, or any organic acid anhydride.

5. A process for preparation of 1,2,2,6,6-pentamethylpiperidin-4-yl methacrylate comprising:
forming a reaction mixture of 1,2,2,6,6-Pentamethyl-4-piperidinol, 4-dimethylaminopyridine and methacrylic anhydride at a temperature between about −20° C. and 10° C.;
warming the reaction mixture to between about 20° C. and 22° C.;
reacting the reaction mixture for between about 15 hours and 24 hours;
quenching the reaction with aqueous saturated NaHCO₃;
purifying a 1,2,2,6,6-pentamethylpiperidin-4-yl methacrylate product from the quenched reaction mixture.

6. The 1,2,2,6,6-pentamethylpiperidin-4-yl methacrylate product of claim 5 having an FTIR spectra of FIG. 2D.

7. The 1,2,2,6,6-pentamethylpiperidin-4-yl methacrylate product of claim 5 having an GC-MS spectra of FIG. 3.

8. The process of claim 5, wherein the reaction mixture is formed with 1,2,2,6,6-pentamethyl-4-piperidinol (50.0 g, 292 mmol), 4-dimethylaminopyridine (4-DMAP, 3.57 g, 29.2 mmol) and methacrylic anhydride (45 g, 292 mmol).

9. The process of claim 1, wherein the reaction mixture is formed at an ideal temperature of about 0° C.

10. The process of claim 5, wherein the step of purifying a 1,2,2,6,6-pentamethylpiperidin-4-yl methacrylate product further comprises:
stirring the quenched reaction product for about 30 minutes;
partitioning the layers and extracting a solution comprising the product;
sequentially washing the solution with aqueous saturated NaHCO₃ (2×300 mL), water (300 mL), brine (300 mL);
drying the washed solution over MgSO₄; and filtering off salts to obtain a purified 1,2,2,6,6-pentamethylpiperidin-4-yl methacrylate product.

11. The process of claim 5, wherein the purified 1,2,2,6,6-pentamethylpiperidin-4-yl methacrylate product is 97% +/−3% active.

12. A UV stabilizing additive package comprising 1,2,2,6,6-pentamethylpiperidin-4-yl methacrylate.

13. The UV stabilizing additive package of claim 12, comprising a blend of 1,2,2,6,6-pentamethylpiperidin-4-yl methacrylate and a second additive.

14. The UV stabilizing additive package of claim 12, comprising a 1,2,2,6,6-pentamethylpiperidin-4-yl methacrylate product having an FTIR spectra of FIG. 2D.

15. The UV stabilizing additive package of claim 12, comprising a 1,2,2,6,6-pentamethylpiperidin-4-yl methacrylate product having an GC-MS spectra of FIG. 3.

16. A thermoset composite comprising 1,2,2,6,6-pentamethylpiperidin-4-yl methacrylate.

17. The thermoset composite of claim 16, comprising a blend of 1,2,2,6,6-pentamethylpiperidin-4-yl methacrylate and a second additive.

18. The thermoset composite of claim 16, comprising 1,2,2,6,6-pentamethylpiperidin-4-yl methacrylate in an amount between about 0.1 phr and 5 phr.

19. The thermoset composite of claim 16, comprising 1,2,2,6,6-pentamethylpiperidin-4-yl methacrylate in an amount of about 2.2 phr.

20. The thermoset composite of claim 16, comprising 1,2,2,6,6-pentamethylpiperidin-4-yl methacrylate prepared by:
forming a reaction mixture of 1,2,2,6,6-pentamethyl-4-piperidinol, 4-dimethylaminopyridine and methacrylic anhydride at a temperature between about −20° C. and 10° C.;
warming the reaction mixture to between about 20° C. and 22° C.;
reacting the reaction mixture for between about 15 hours and 24 hours;
quenching the reaction with aqueous saturated NaHCO₃;
purifying a 1,2,2,6,6-pentamethylpiperidin-4-yl methacrylate product from the quenched reaction mixture.
